(19) United States
(12) Patent Application Publication
(10) Pub. No.: US 2025/0032457 A1
(43) Pub. Date: Jan. 30, 2025

(54) METHODS OF TREATING ANDROGENIC ALOPECIA AND/OR SKIN CONDITIONS

(71) Applicant: Eric Rauch, San Pedro, CA (US)

(72) Inventor: Eric Rauch, San Pedro, CA (US)

(21) Appl. No.: 18/838,889

(22) PCT Filed: Sep. 14, 2021
(86) PCT No.: PCT/US2021/050320

§ 371 Date: Aug. 15, 2024

Related U.S. Application Data
(60) Provisional application No. 63/078,803, filed on Sep. 15, 2020.

(51) Int. Cl. A61K 31/4174; A61K 8/49; A61K 31/4164; A61K 31/506; A61K 31/56; A61P 17/14; A61Q 7/00; A61Q 19/08

(52) U.S. Cl. CPC A61K 31/4174; A61K 8/4946; A61K 31/4164; A61K 31/506; A61K 31/56; A61P 17/14; A61Q 7/00; A61Q 19/08

(57) ABSTRACT

Described herein are methods of using one or more azole compounds in treating androgenic alopecia, reducing or stopping hair thinning, reversing hair thinning, promoting hair growth, reversing hairline recession, and/or treating a skin condition.

### CROSS-REFERENCE TO RELATED APPLICATIONS

This application claims the benefit of U.S. Provisional Application No. 63/078,803, filed Sep. 15, 2020, the content of which is incorporated by reference herein in its entirety.

### BACKGROUND

The present technology relates generally to the field of formulations for reducing, stopping or reversing hair thinning or hair loss, or treating skin conditions.

### SUMMARY

In one aspect, provided herein is a method of treating androgenic alopecia in a subject in need thereof, the method comprising administering to the subject a therapeutically effective amount of econazole, prochloraz, or a combination thereof.

In another aspect, provided herein is a method of reducing or stopping hair thinning in a subject in need thereof, the method comprising administering to the subject a therapeutically effective amount of econazole, prochloraz, or a combination thereof.

In another aspect, provided herein is a method of reversing hair thinning in a subject in need thereof, the method comprising administering to the subject a therapeutically effective amount of econazole, prochloraz, or a combination thereof.

In another aspect, provided herein is a method of promoting hair growth in a subject in need thereof, the method comprising administering to the subject a therapeutically effective amount of econazole, prochloraz, or a combination thereof.

In some embodiments, econazole, prochloraz, or the combination thereof is administered topically on a scalp of the subject. In some embodiments, the scalp is shaved prior to administration of econazole, prochloraz, or the combination thereof. In some embodiments, the scalp is shaved 2-3 times weekly for 4 to 6 weeks upon commencement of administration of econazole, prochloraz, or the combination thereof.

In some embodiments, the subject is a human. In some embodiments, the subject is a non-human mammal.

In another aspect, provided herein is a method of reversing hairline recession in a subject in need thereof, the method comprising administering to the subject a therapeutically effective amount of econazole or prochloraz, or a combination thereof. In some embodiments, the method includes reversing bitemporal recession. In some embodiments, econazole, prochloraz, or the combination thereof is administered topically at an area of scalp with hairline recession. In some embodiments, the scalp is shaved prior to administration of econazole, prochloraz, or the combination thereof. In some embodiments, the scalp is shaved 2-3 times weekly for 4 to 6 weeks upon commencement of administration of econazole, prochloraz, or the combination thereof.

In another aspect, provided herein is a method of treating a skin condition in a subject in need thereof, the method comprising administering to the subject a therapeutically effective amount of econazole or prochloraz, or a combination thereof. In some embodiments, the skin condition is skin aging or one or more selected from the group consisting of dry skin, psoriasis, eczema, acne, seborrheic dermatitis, wrinkling, and loss of collagen. In some embodiments, econazole, prochloraz, or the combination thereof is administered topically at an area of skin with skin aging or one or more selected from the group consisting of dry skin, psoriasis, eczema, acne, seborrheic dermatitis, wrinkling, and loss of collagen. In some embodiments, the area of skin is located at the face, neck, chest, back, or any combination of two or more thereof.

Further provided herein is a composition comprising, or consisting essentially of, or yet further consisting of an effective amount of econazole, prochloraz, or a combination thereof and a carrier, such as for example a pharmaceutically acceptable carrier.

In some embodiments, econazole, prochloraz, or the combination thereof is administered as a formulation selected from a cream, ointment, lotion, serum, foam, gel, paste, aerosol, powder, or liquid. In some embodiments, econazole, prochloraz, or the combination thereof, is present in the formulation in an amount of about 0.1 wt. % to about 10 wt. %. In some embodiments, econazole, prochloraz, or the combination thereof are formulated for administration through immersion of, bathing, or soaking the subject.

In some embodiments, econazole, prochloraz, or the combination thereof is administered at least once per day. In some embodiments, econazole, prochloraz, or the combination thereof is administered once or twice per day

In some embodiments, econazole, prochloraz, or the combination thereof is administered in combination with one or more additional therapeutic agents. In some embodiments, the one or more additional therapeutic agents are selected from minoxidil, finasteride, dutasteride another azole compound, and any combination of two or more thereof. In some embodiments, econazole, prochloraz, or the combination thereof and the one or more additional therapeutic agents are administered concurrently. In some embodiments, econazole, prochloraz, or the combination thereof and the one or more additional therapeutic agents are administered sequentially. Compositions and formulations for the combination therapy are further provided herein, each having an effective amount of the active agent for the purpose of the therapy.

### BRIEF DESCRIPTION OF THE DRAWINGS

FIG. 1 depicts a picture of a human male subject prior to topical administration with an azole compound. Hairline recession, hair thinning, and hair loss were observed prior to administration.

FIG. 2 depicts a picture of a human male subject after topical administration with an azole compound. Hairline recession, hair thinning, and hair loss were reversed.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “subject” refers to an animal, such as a mammal (including a human), that has been or will be the object of treatment, observation or experiment. “Subject” and “patient” may be used interchangeably, unless otherwise indicated. Mammals include, but are not limited to, mice, rodents, rats, simians, humans, farm animals, dogs, cats, sport animals, and pets. The methods described herein may be useful in human therapy and/or veterinary applications such as but limited to pet grooming. In some embodiments, the subject is a mammal. In some embodiments, the subject is a human. In some embodiments, the human is male. In some embodiments, the human is female. In some embodiments, the human is male or female.

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease or disorder, or the manner of administration as determined by a qualified prescriber or caregiver. In terms of the composition, the effective amount will vary with the treatment and the patient. As such, the compositions can be formulated by a compounding pharmacy for personalized therapy of the disease being treated provided in a standard range for therapeutic use.

The term “treatment” or “treating” means administering a formulation disclosed herein for the purpose of: (i) delaying the onset of a disease/disorder, that is, causing the clinical symptoms of the disease/disorder not to develop or delaying the development thereof, (ii) inhibiting the disease/disorder, that is, arresting the development of clinical symptoms; and/or (iii) relieving the disease/disorder, that is, causing the regression of clinical symptoms or the severity thereof. In one aspect, the term “treatment” excludes inhibition or prophylaxis of the disease/disorder.

By “pharmaceutically acceptable” is meant a material that is not biologically or otherwise undesirable, e.g., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. A pharmaceutically acceptable salt of an active agent can be used instead of the free base form of the active agent in any formulation disclosed herein. In a further aspect, the compositions can optionally comprise a penetration enhancer which are known in the art.

As used herein, the term “administration” and “administering” are used to mean introducing an agent into a subject. Routes of administration include, but are not limited to, oral (such as a tablet, capsule or suspension), topical, transdermal, intradermal, intranasal, vaginal, rectal, subcutaneous intravenous, intravenous, intraarterial, intramuscular, intraosseous, intraperitoneal, intraocular, subconjunctival, sub-Tenon's, intravitreal, retrobulbar, intracameral, intratumoral, epidural and intrathecal.

Unless defined otherwise, all technical and scientific terms used herein have the same meaning as commonly understood by one of ordinary skill in the art to which this present invention belongs. Although any methods and materials similar or equivalent to those described herein can also be used in the practice or testing of the present invention, representative illustrative methods and materials are described herein.

### Hair Loss and Hair Thinning

Hair loss and hair thinning may be the result of heredity, hormonal changes, medical conditions, or medical procedures, such as chemotherapy or radiation therapy.

Androgenic alopecia is a hereditary condition that commonly occurs with aging, resulting in hair loss. Often referred to as male-pattern baldness or female-pattern baldness, androgenic alopecia occurs gradually and in predictable patterns. With male-pattern baldness, hair loss typically occurs on the top and front of the head. With female-pattern baldness, thinning occurs on the top and crown of the head. Presence and severity of bitemporal recession does not correlate with vertex or mid-frontal scalp hair loss.

Treatments of hair loss and hair thinning include minoxidil, finasteride, or hair transplant surgery. Minoxidil and finasteride have been clinically shown to regrow hair on the top of the scalp (i.e., vertex region). However, minoxidil is not intended for use to treat frontal baldness nor receding hairline, and efficacy of finasteride in bitemporal recession has not been established. In addition, termination of treatment with minoxidil and finasteride generally leads to recommencement of hair loss. Hair transplant surgery is costly and may have unpredictable results. Accordingly, alternative treatments are needed.

### Methods of Use

In one aspect, provided herein are methods of treating androgenic alopecia in a subject in need thereof, the method comprising, consisting essentially of, or consisting of administering to the subject a therapeutically effective amount of an azole compound. In some embodiments, the azole compound is administered topically on a scalp of the subject.

In another aspect, provided herein are methods of reducing or stopping hair thinning in a subject in need thereof, the method comprising, consisting essentially of, or consisting of administering to the subject a therapeutically effective amount of an azole compound. In some embodiments, the azole compound is administered topically on a scalp of the subject.

In another aspect, provided herein are methods of reducing hair thinning in a subject in need thereof, the method comprising, consisting essentially of, or consisting of administering to the subject a therapeutically effective amount of an azole compound. In some embodiments, the azole compound is administered topically on a scalp of the subject.

In another aspect, provided herein are methods of stopping hair thinning in a subject in need thereof, the method comprising, consisting essentially of, or consisting of administering to the subject a therapeutically effective amount of an azole compound. In some embodiments, the azole compound is administered topically on a scalp of the subject.

In another aspect, provided herein are methods of reversing hair thinning in a subject in need thereof, the method comprising, consisting essentially of, or consisting of administering to the subject a therapeutically effective amount of an azole compound. In some embodiments, the azole compound is administered topically on a scalp of the subject.

In another aspect, provided herein are methods of promoting hair growth in a subject in need thereof, the method comprising, consisting essentially of, or consisting of administering to the subject a therapeutically effective amount of an azole compound. In some embodiments, the azole compound is administered topically on a scalp of the subject.

In another aspect, provided herein are methods of treating androgenic alopecia in a subject in need thereof, the method comprising, consisting essentially of, or consisting of administering to the subject a therapeutically effective amount of econazole, prochloraz, or a combination thereof. In some embodiments, econazole, prochloraz, or the combination thereof is administered topically on a scalp of the subject.

In another aspect, provided herein are methods of reducing or stopping hair thinning in a subject in need thereof, the method comprising, consisting essentially of, or consisting of administering to the subject a therapeutically effective amount of econazole, prochloraz, or a combination thereof. In some embodiments, econazole, prochloraz, or the combination thereof is administered topically on a scalp of the subject.

In another aspect, provided herein are methods of reducing hair thinning in a subject in need thereof, the method comprising, consisting essentially of, or consisting of administering to the subject a therapeutically effective amount of econazole, prochloraz, or a combination thereof. In some embodiments, econazole, prochloraz, or the combination thereof is administered topically on a scalp of the subject.

In another aspect, provided herein are methods of stopping hair thinning in a subject in need thereof, the method comprising, consisting essentially of, or consisting of administering to the subject a therapeutically effective amount of econazole, prochloraz, or a combination thereof. In some embodiments, econazole, prochloraz, or the combination thereof is administered topically on a scalp of the subject.

In another aspect, provided herein are methods of reversing hair thinning in a subject in need thereof, the method comprising, consisting essentially of, or consisting of administering to the subject a therapeutically effective amount of econazole, prochloraz, or a combination thereof. In some embodiments, econazole, prochloraz, or the combination thereof is administered topically on a scalp of the subject.

In another aspect, provided herein are methods of promoting hair growth in a subject in need thereof, the method comprising, consisting essentially of, or consisting of administering to the subject a therapeutically effective amount of econazole, prochloraz, or a combination thereof. In some embodiments, econazole, prochloraz, or the combination thereof is administered topically on a scalp of the subject.

The scalp of the subject includes the temporal, frontal, vertex, parietal, occipital, and nuchal regions. In some embodiments of the methods described herein, econazole, prochloraz, or the combination thereof is administered topically on one or more of the temporal, frontal, vertex, parietal, occipital, and nuchal regions of the scalp. In some embodiments of the methods described herein, the azole compound is administered topically on one or both temporal regions of the scalp. In some embodiments of the methods described herein, the azole compound is administered topically on the frontal region of the scalp. In some embodiments of the methods described herein, the azole compound is administered topically on the vertex region of the scalp. In some embodiments of the methods described herein, the azole compound is administered topically on the parietal region of the scalp. In some embodiments of the methods described herein, the azole compound is administered topically on the occipital region of the scalp. In some embodiments of the methods described herein, econazole, the azole compound is administered topically on the nuchal region of the scalp.

In some embodiments, the azole compound is administered to skin, wherein the skin has active hair follicles. In some embodiments, the azole compound is administered to skin, wherein the skin has vellus hairs, i.e. “peach fuzz”. In some embodiments, the azole compound is administered to skin, wherein the skin is smooth with no apparent hair growth as observed by the naked eye. In some embodiments, the methods as described herein further comprise shaving or dermabrasion of the skin to be treated immediately prior to administering the azole compound to the skin. In some embodiments, the method further comprises shaving or dermabrasion of the skin 2-3 times per week for 4-6 weeks after initially administering the azole compound to the skin.

In another aspect, provided herein are methods of reversing hairline recession in a subject in need thereof, the method comprising, consisting essentially of, or consisting of administering to the subject a therapeutically effective amount of an azole compound. In some embodiments, the methods include reversing bitemporal recession. In some embodiments, the azole compound is administered topically at an area of scalp with hairline recession. In some embodiments, “hairline” refers to the edge of the area of the scalp where hair grows. In further embodiments, hairline recession leads to the hairline moving further away from the face, typically affecting the frontal or one or more temporal regions of the scalp, or a combination of two or more thereof. As used herein, unless otherwise indicated, “reversing hairline recession” refers to movement of the hairline towards the face. Reversing hairline recession may be evaluated with the naked eye by the comparison of photographs before and after administration of an azole compound, as described herein.

In another aspect, provided herein are methods of reversing bitemporal recession in a subject in need thereof, the method comprising, consisting essentially of, or consisting of administering to the subject a therapeutically effective amount of an azole compound. In some embodiments, the azole compound is administered topically at an area of scalp with bitemporal recession.

In another aspect, provided herein are methods of reversing hairline recession in a subject in need thereof, the method comprising, consisting essentially of, or consisting of administering to the subject a therapeutically effective amount of econazole, prochloraz, or a combination thereof. In some embodiments, the methods include reversing bitemporal recession. In some embodiments, econazole, prochloraz, or the combination thereof is administered topically at an area of scalp with hairline recession.

In another aspect, provided herein are methods of reversing bitemporal recession in a subject in need thereof, the method comprising, consisting essentially of, or consisting of administering to the subject a therapeutically effective amount of econazole, prochloraz, or a combination thereof. In some embodiments, econazole, prochloraz, or the combination thereof is administered topically at an area of scalp with bitemporal recession.

In another aspect, provided herein are methods of treating a skin condition in a subject in need thereof, the method comprising, consisting essentially of, or consisting of administering to the subject a therapeutically effective amount of an azole compound.

In another aspect, provided herein are methods of treating a skin condition in a subject in need thereof, the method comprising, consisting essentially of, or consisting of administering to the subject a therapeutically effective amount of econazole or prochloraz, or a combination thereof.

In some embodiments, the skin condition is one or more selected from the group of skin aging, dry skin, psoriasis, eczema, acne, seborrheic dermatitis, wrinkling (including premature wrinkling), or loss of collagen. In some embodiments, the skin condition is one or more selected from the group consisting of acne, seborrheic dermatitis, wrinkling, and loss of collagen. In some embodiments, the skin condition is acne. In some embodiments, the skin condition is seborrheic dermatitis. In some embodiments, the skin condition is wrinkling. In some embodiments, the skin condition is loss of collagen. In some embodiments, the skin condition is dry skin. In some embodiments, the skin condition is psoriasis. In some embodiments, the skin condition is eczema.

In some embodiments, the skin condition is skin aging. As used herein, “skin aging” refers to one or more of the following features in skin: wrinkling, laxity (i.e., loose skin), and rough-textured appearance.

In some embodiments, the azole compound (e.g., econazole or prochloraz, or a combination thereof) is administered topically at an area of skin with the skin condition. In some embodiments, the area of skin is located at the face, neck, chest, back, or any combination of two or more thereof. In some embodiments, the area of skin is located at the face. In some embodiments, the area of skin is located at the neck. In some embodiments, the area of skin is located at the chest. In some embodiments, the area of skin is located at the back.

### Azole Compounds and Compositions Thereof

Azole compounds may be categorized as imidazole or triazole compounds. Non-limiting examples of azole compounds include econazole, prochloraz, ketoconazole, miconazole, fluconazole, itraconazole, voriconazole, posaconazole, clotrimazole, oxiconazole, terconizole, tioconazole, T-8581, ravuconazole, saperconazole, D0870, UR-9746, UR-9751, TAK187, SSY 726, KP-103, luliconazole, lanoconazole, clotrimazole, bifonazole, enilconazole, genaconazole, epoxiconazole, propiconazole, and tebuconazole. Azole compounds may be commercially available from companies, e.g., Sigma-Aldrich (www.sigmaaldrich.com; last accessed Sep. 14, 2020) and Fisher Scientific (www.fishersci.com; last accessed Sep. 14, 2020), and/or prepared using methods known to one of ordinary skill in the art.

The azole compound is formulated as a pharmaceutically acceptable composition when combined with at least one pharmaceutically acceptable carrier and/or excipient. Such pharmaceutically acceptable carrier(s) and/or excipient(s) are non-toxic and do not interfere with the efficacy of active ingredient (e.g., the azole compound, optional one or more additional therapeutic agents).

The azole compound may be administered in the form of its pharmaceutically acceptable salt (such as, but not limited to, an acetate salt) and/or as a pharmaceutically acceptable solvate of the salt thereof or of the free base form thereof.

Examples of excipients include, but are not limited to, a stabilizer, a surfactant (such as a nonionic, ionic, anionic, cationic, or zwitterionic surfactant), a salt and/or a buffering agent, solubilizer (such as glycerol or PEG 40 castor oil), an antioxidant, an anesthetic agent, a penetration enhancer, an anesthetic agent (such as a morphine derivative), a thickening agent, an emulsifier, a chelating agent, and a preservative.

In some embodiments, the stabilizer may, for example, be an amino acid, such as for instance, glycine or an oligosaccharide, such as for example, sucrose, tetralose, lactose or a dextran. Alternatively, the stabilizer may be a sugar alcohol, such as for instance, mannitol, sorbitol, xylitol, or a combination thereof. In some embodiments, the stabilizer or combination of stabilizers constitutes from about 0.1% to about 10% by weight of the formulation, or any percentage in between these two values.

In some embodiments, the surfactant is a nonionic surfactant, such as a polysorbate. Some examples of suitable surfactants include polysorbates (e.g., Tween20, Tween80); and a polyethylene glycol or a polyoxyethylene polyoxypropylene glycol, such as Pluronic F-68 at from about 0.001% (w/v) to about 10% (w/v), or any percentage in between these two values.

A salt or buffering agent may be any salt or buffering agent, such as for example, sodium chloride, or sodium/potassium phosphate, respectively. In certain embodiments, the buffering agent maintains the pH of the pharmaceutical composition in the range of about 5.5 to about 7.5, or any pH in between these two values. The salt and/or buffering agent is also useful to maintain the osmolality at a level suitable for administration to a human or an animal. In some embodiments, the salt or buffering agent is present at a roughly isotonic concentration of about 150 mM to about 300 mM. Other buffering agents include, but are not limited to, triethanolamine, sodium hydroxide, potassium hydroxide, cocoamidodiethylamine, and any combination thereof.

Non-limiting examples of an antioxidant include benzalkonium chloride (a mixture of quaternary ammonium compounds, known as “quats”), benzyl alcohol, caprylic/capric triglyceride, chloretone, and chlorobutanol.

Non-limiting examples of a penetration enhancer include diethylene glycol monoethyl ether, lauryl alcohol, dimethyl sulfoxide (DMSO), propylene glycol, dimethyl acetamide, N-methyl pyrolidone, oleic acid, azone, oxazolidinone derivatives, urea, terpenes (including, but not limited to, menthol, linalyl alcohol, eugenol, limonene, pinene, and squalene), and any combination thereof.

Non-limiting examples of a thickening agent include a cross-linked polyacrylic acid polymer (e.g, a carbomer), a cellulose derivative (e.g., hydroxyethylcellulose, ethyl cellulose, hydroxypropyl cellulose, carboxymethyl cellulose, sodium carboxymethyl cellulose), xanthan gum, locust beam gum, guar gum or derivative thereof, and alginic acid.

Non-limiting examples of an emulsifier include ceteareth-20, glyceryl stearate, PEG-100 stearate, and stearic acid.

Non-limiting examples of a chelating agent include EDTA or a salt or solvate thereof, such as disodium EDTA.

Non-limiting examples of a preservative include phenoxyethanol, urea derivatives (such as, but not limited to, diazolidinyl urea and imidazolidinyl urea), ethylhexylglycerine, hydantoin, benzoic acid, sorbic acid, anisic acid, methylparaben, propylparaben, and any combination thereof.

The formulation may also include water and/or one or more other solvents, such as an alcohol (e.g., ethanol).

In some embodiments, petrolatum is included in the formulation as an ointment base or a cream base.

The amount of any individual excipient in the composition will vary depending on the role of the excipient, the dosage requirements of the active agent components, and particular needs of the composition. Generally, however, the excipient will be present in the composition in an amount of about 1% to about 99% by weight, from about 5% to about 98% by weight, or from about 15 to about 95% by weight of the excipient. In general, the amount of excipient present in a composition of the disclosure is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% or more by weight.

In any embodiments, the azole compound (e.g., econazole or prochloraz, or a combination thereof) is administered as a formulation in the form of a topical formulation. Topical formulations include, but are not limited to, gels, lotions, creams, ointments, pastes, serums, foams, aerosols, powders, or liquids (e.g., suspension or solution). In any embodiments, the topical formulation may be a semi-solid formulation. A semi-solid formulation includes, but is not limited to, a gel, a lotion, a cream, an ointment, a suspension, a paste, a serum, and a foam.

In some embodiments, the formulation disclosed herein may be in the form of a lotion, cream, gel, paste, serum, or ointment. In some embodiments, the formulation disclosed herein is a cream. In some embodiments, the formulation disclosed herein is an ointment. In some embodiments, the formulation disclosed herein is a lotion. In some embodiments, the formulation disclosed herein is a liquid. In some embodiments, the formulation disclosed herein is a paste. In some embodiments, the formulation disclosed herein is a gel. In some embodiments, the formulation disclosed herein is a foam. In some embodiments, the formulation disclosed herein is an aerosol. In some embodiments, the formulation disclosed herein is a powder. In some embodiments, the formulation is suitable for immersion, bathing, or soaking, such as, but not limited to, in a spa treatment. Non-limiting examples of formulations suitable for immersion, bathing, or soaking include solutions, suspensions, gels, or foams that may be dissolved or dispersed in water.

In some embodiments, the azole compound (e.g., econazole or prochloraz, or a combination thereof) is present in the formulation in an amount of about 0.1 wt. % to about 10 wt. % (weight percent with respect to the formulation). This includes about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 wt. %, including increments therein.

In some embodiments, the azole compound (e.g., econazole or prochloraz, or a combination thereof) is present in the formulation in an amount of about 0.1 wt. % to about 9 wt. %, about 0.1 wt. % to about 8 wt. %, about 0.1 wt. % to about 7 wt. %, about 0.1 wt. % to about 6 wt. %, about 0.1 wt. % to about 5 wt. %, about 0.1 wt. % to about 4 wt. %, about 0.1 wt. % to about 3 wt. %, about 0.1 wt. % to about 2 wt. %, about 0.1 wt. % to about 1 wt. %, about 0.2 wt. % to about 5 wt. %, about 0.2 wt. % to about 4 wt. %, about 0.2 wt. % to about 3 wt. %, about 0.2 wt. % to about 2 wt. %, about 0.2 wt. % to about 1 wt. %, about 0.3 wt. % to about 5 wt. %, about 0.3 wt. % to about 4 wt. %, about 0.3 wt. % to about 3 wt. %, about 0.3 wt. % to about 2 wt. %, about 0.3 wt. % to about 1 wt. %, about 0.4 wt. % to about 5 wt. %, about 0.4 wt. % to about 4 wt. %, about 0.4 wt. % to about 3 wt. %, about 0.4 wt. % to about 2 wt. %, about 0.4 wt. % to about 1 wt. %, about 0.5 wt. % to about 5 wt. %, about 0.5 wt. % to about 4 wt. %, about 0.5 wt. % to about 3 wt. %, about 0.5 wt. % to about 2 wt. %, about 0.5 wt. % to about 1 wt. %, about 0.7 wt. % to about 5 wt. %, about 0.7 wt. % to about 4 wt. %, about 0.7 wt. % to about 3 wt. %, about 0.7 wt. % to about 2 wt. %, about 0.7 wt. % to about 1 wt. %, about 1 wt. % to about 5 wt. %, about 1 wt. % to about 4 wt. %, about 1 wt. % to about 3 wt. %, about 1 wt. % to about 2 wt. %, about 2 wt. % to about 5 wt. %, about 2 wt. % to about 4 wt. %, about 2 wt. % to about 3 wt. %, about 3 wt. % to about 5 wt. %, about 3 wt. % to about 4 wt. %, about 4 wt. % to about 5 wt. %, about 1 wt. % to about 10 wt. %, or about 0.5 wt. % to about 10 wt. %.

In any embodiments, the azole compound (e.g., econazole or prochloraz, or a combination thereof) may be present in the formulation in an amount of about 1 mg to about 5000 mg. This includes about 1 mg to about 4500 mg, about 1 mg to about 4000 mg, about 1 mg to about 3500 mg, about 1 mg to about 3000 mg, about 1 mg to about 2500 mg, about 1 mg to about 2000 mg, about 1 mg to about 1500 mg, about 1 mg to about 1000 mg, about 1 mg to about 750 mg, about 1 mg to about 500 mg, about 1 mg to about 400 mg, about 1 mg to about 300 mg, about 1 mg to about 200 mg, about 1 mg to about 100 mg, about 1 mg to about 75 mg, about 1 mg to about 50 mg, about 1 mg to about 25 mg, about 5 mg to about 400 mg, about 5 mg to about 300 mg, about 5 mg to about 200 mg, about 5 mg to about 100 mg, about 5 mg to about 75 mg, about 5 mg to about 50 mg, about 10 mg to about 400 mg, about 10 mg to about 300 mg, about 10 mg to about 200 mg, about 10 mg to about 100 mg, about 10 mg to about 75 mg, about 10 mg to about 50 mg, about 25 mg to about 400 mg, about 25 mg to about 300 mg, about 25 mg to about 200 mg, about 25 mg to about 100 mg, about 25 mg to about 75 mg, about 25 mg to about 50 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 250 mg, about 50 mg to about 275 mg, about 50 mg to about 300 mg, about 50 mg to about 325 mg, about 50 mg to about 350 mg, about 50 mg to about 375 mg, about 50 mg to about 400 mg, about 50 mg to about 425 mg, about 50 mg to about 450 mg, about 50 mg to about 475 mg; about 100 mg to about 125 mg, about 100 mg to about 150 mg, about 100 mg to about 175 mg, about 100 mg to about 200 mg, about 100 mg to about 225 mg, about 100 mg to about 250 mg, about 100 mg to about 275 mg, about 100 mg to about 300 mg, about 100 mg to about 325 mg, about 100 mg to about 350 mg, about 100 mg to about 375 mg, about 100 mg to about 400 mg, about 100 mg to about 425 mg, about 100 mg to about 450 mg, about 100 mg to about 475 mg, about 100 mg to about 500 mg, about 100 mg to about 1000 mg, about 100 mg to about 2000 mg, about 100 mg to about 3000 mg, about 100 mg to about 4000 mg, about 100 mg to about 5000 mg, about 250 mg to about 1000 mg, about 250 mg to about 2000 mg, about 250 mg to about 3000 mg, about 250 mg to about 4000 mg, about 250 mg to about 5000 mg, about 500 mg to about 1000 mg, about 500 mg to about 2000 mg, about 500 mg to about 3000 mg, about 500 mg to about 4000 mg, about 500 mg to about 5000 mg, about 750 mg to about 1000 mg, about 750 mg to about 2000 mg, about 750 mg to about 3000 mg, about 750 mg to about 4000 mg, about 750 mg to about 5000 mg, about 1000 mg to about 2000 mg, about 1000 mg to about 3000 mg, about 1000 mg to about 4000 mg, and about 1000 mg to about 5000 mg. Thus, the azole compound (e.g., econazole or prochloraz, or a combination thereof) may be present in the formulation disclosed herein in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000, 1250, 1500, 1750, 2000, 2250, 2500, 2750, 3000, 3250, 3500, 3750, 4000, 4250, 4500, 4750, or 5000 mg, including increments therein.

In any embodiments, the azole compound (e.g., econazole or prochloraz, or a combination thereof) may be present in the formulation disclosed herein in an amount of about 1 mg/mL to about 500 mg/mL. This includes about 1 mg/mL to about 25 mg/mL; about 1 mg/mL to about 50 mg/mL; about 1 mg/mL to about 75 mg/mL; about 1 mg/mL to about 100 mg/mL, 1 mg/mL to about 125 mg/mL, 1 mg/mL to about 150 mg/mL, 1 mg/mL to about 175 mg/mL, 1 mg/mL to about 200 mg/mL, 1 mg/mL to about 225 mg/mL, 1 mg/mL to about 250 mg/mL, 1 mg/mL to about 275 mg/mL, 1 mg/mL to about 300 mg/mL, 1 mg/mL to about 325 mg/mL, 1 mg/mL to about 350 mg/mL, 1 mg/mL to about 375 mg/mL, 1 mg/mL to about 400 mg/mL, 1 mg/mL to about 425 mg/mL, 1 mg/mL to about 450 mg/mL, 1 mg/mL to about 475 mg/mL; about 25 mg/mL to about 50 mg/mL, about 25 mg/mL to about 75 mg/mL, about 25 mg/mL to about 100 mg/mL, 25 mg/mL to about 125 mg/mL, 25 mg/mL to about 150 mg/mL, 25 mg/mL to about 175 mg/mL, 25 mg/mL to about 200 mg/mL, 25 mg/mL to about 225 mg/mL, 25 mg/mL to about 250 mg/mL, 25 mg/mL to about 275 mg/mL, 25 mg/mL to about 300 mg/mL, 25 mg/mL to about 325 mg/mL, 25 mg/mL to about 350 mg/mL, 25 mg/mL to about 375 mg/mL, 25 mg/mL to about 400 mg/mL, 25 mg/mL to about 425 mg/mL, 25 mg/mL to about 450 mg/mL, 25 mg/mL to about 475 mg/mL; about 50 mg/mL to about 100 mg/mL, 50 mg/mL to about 125 mg/mL, 50 mg/mL to about 150 mg/mL, 50 mg/mL to about 175 mg/mL, 50 mg/mL to about 200 mg/mL, 50 mg/mL to about 225 mg/mL, 50 mg/mL to about 250 mg/mL, 50 mg/mL to about 275 mg/mL, 50 mg/mL to about 300 mg/mL, 50 mg/mL to about 325 mg/mL, 50 mg/mL to about 350 mg/mL, 50 mg/mL to about 375 mg/mL, 50 mg/mL to about 400 mg/mL, 50 mg/mL to about 425 mg/mL, 50 mg/mL to about 450 mg/mL, 50 mg/mL to about 475 mg/mL; 100 mg/mL to about 125 mg/mL, 100 mg/mL to about 150 mg/mL, 100 mg/mL to about 175 mg/mL, 100 mg/mL to about 200 mg/mL, 100 mg/mL to about 225 mg/mL, 100 mg/mL to about 250 mg/mL, 100 mg/mL to about 275 mg/mL, 100 mg/mL to about 300 mg/mL, 100 mg/mL to about 325 mg/mL, 100 mg/mL to about 350 mg/mL, 100 mg/mL to about 375 mg/mL, 100 mg/mL to about 400 mg/mL, 100 mg/mL to about 425 mg/mL, 100 mg/mL to about 450 mg/mL, 100 mg/mL to about 475 mg/mL, and 100 mg/mL to about 500 mg/mL. Thus, econazole, prochloraz, or the combination thereof, may be present in the formulation disclosed herein in an amount of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/mL, including increments therein.

In some embodiments, the azole compound (e.g., econazole or prochloraz, or a combination thereof) is administered at least once per day. In some embodiments, the azole compound (e.g., econazole or prochloraz, or a combination thereof) is administered at least twice per day. In some embodiments, the azole compound (e.g., econazole or prochloraz, or a combination thereof) is administered once or twice per day. In some embodiments, the azole compound (e.g., econazole or prochloraz, or a combination thereof) is administered once per day. In some embodiments, the azole compound (e.g., econazole or prochloraz, or a combination thereof) is administered twice per day.

### Additional Therapeutic Agents

In some embodiments, the azole compound is administered in combination with one or more additional therapeutic agents. In some embodiments, the azole compound and the one or more additional therapeutic agents are administered concurrently. In some embodiments, the azole compound and the one or more additional therapeutic agents are administered sequentially.

In some embodiments, the azole compound and the one or more additional therapeutic agents are administered as a single composition. In some embodiments, the azole compound and the one or more additional therapeutic agents are administered as a single composition. In some embodiments, the azole compound and the one or more additional therapeutic agents are administered as a single composition. In some embodiments, the azole compound and the one or more additional therapeutic agents are administered as two or more compositions.

In some embodiments, the one or more additional therapeutic agents are selected from another azole compound, minoxidil, finasteride, dutasteride, or fenugreek (Trigonella foenum-graecum) or any combination of two or more thereof. In some embodiments, the one or more additional therapeutic agents are selected from another azole compound, minoxidil, finasteride, and dutasteride or any combination of two or more thereof. In some embodiments, the one or more additional therapeutic agents is minoxidil. In some embodiments, the one or more additional therapeutic agents is finasteride. In some embodiments, the one or more additional therapeutic agents is fenugreek. In some embodiments, fenugreek is administered orally or topically. In some embodiments, fenugreek is administered as an oral supplement. In some embodiments, fenugreek is administered as a formulation suitable for immersion, bathing, or soaking.

In some embodiments, the azole compound is econazole or prochloraz, or a combination thereof, and the one or more additional therapeutic agents are selected from minoxidil, finasteride, dutasteride, ketoconazole, and miconazole. In some embodiments, the one or more additional therapeutic agents are selected from minoxidil, finasteride and dutasteride. In some embodiments, the one or more additional therapeutic agents are selected from ketoconazole and miconazole. In some embodiments, the one or more additional therapeutic agents is minoxidil. In some embodiments, the one or more additional therapeutic agents is finasteride or dutasteride. In some embodiments, the one or more additional therapeutic agents is ketoconazole. In some embodiments, the one or more additional therapeutic agents is miconazole.

In some embodiments, the azole compound is econazole or prochloraz, or a combination thereof, and the one or more additional therapeutic agents are selected from epoxiconazole, propiconazole, and tebuconazole. In some embodiments, the one or more additional therapeutic agents is epoxiconazole. In some embodiments, the one or more additional therapeutic agents is propiconazole. In some embodiments, the one or more additional therapeutic agents is tebuconazole.

The one or more additional therapeutic agents may be administered using any administrative route, including but not limited to, oral, parenteral, pulmonary, topical, rectal, nasal, vaginal administration, or via implanted reservoir. Parenteral or systemic administration includes, but is not limited to, subcutaneous, intravenous, intraperitoneally, intramuscular, intra-articular, intrasynovial, intrasternal, intrathecal, intralesional and intracranial injections.

Econazole and the one or more additional therapeutic agents may be obtained from a compounding pharmacy.

In some embodiments, one or more compositions disclosed herein are contained in a kit. Accordingly, in some embodiments, provided herein is a kit comprising, consisting essentially of, or consisting of one or more compositions disclosed herein and instructions for their use.

The present invention, thus generally described, will be understood more readily by reference to the following examples, which are provided by way of illustration and are not intended to be limiting of the present invention.

### EXAMPLES

### Example 1

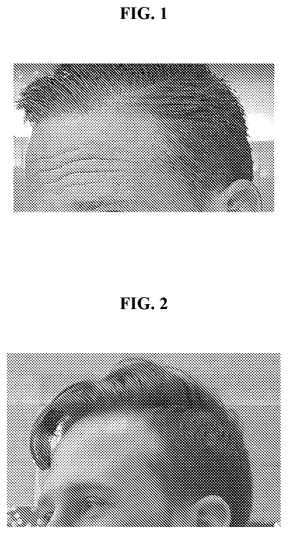

A formulation of miconazole (2% miconazole nitrate) was topically administered twice per day for 9 months to the scalp of a human male subject who prior to administration exhibited hairline recession and hair loss and thinning on the scalp, including frontal, vertex and parietal regions (FIG. 1). After 3 months, reversal of hairline recession and promotion of hair growth elsewhere on the scalp was observed (FIG. 2). Results were found to be superior to those obtained through previous administration of minoxidil (5%) or finasteride (1.25 mg daily) on same human subject, including both in combination.

### Example 2

A formulation of econazole is topically administered at least once per day for up to six months to the scalp of a human subject who prior to administration exhibits hair loss and/or thinning on the scalp.

### Example 3

A formulation of prochloraz is topically administered at least once per day for up to six months to the scalp of a human subject who prior to administration exhibits hair loss and/or thinning on the scalp.

### Example 4

A formulation of econazole and prochloraz is topically administered at least once per day for up to six months to the scalp of a human subject who prior to administration exhibits hair loss and/or thinning on the scalp.

### Example 5

A formulation of econazole is topically administered at least once per day for up to six months, in combination with one or more selected from minoxidil, finasteride, dutasteride and another azole compound, to the scalp of a human subject who prior to administration exhibits hair loss and/or thinning on the scalp.

### Example 6

A formulation of prochloraz is topically administered at least once per day for up to six months, in combination with one or more selected from minoxidil, finasteride, dutasteride and another azole compound, to the scalp of a human subject who prior to administration exhibits hair loss and/or thinning on the scalp.

### Example 7

A formulation of econazole and prochloraz is topically administered at least once per day for up to six months, in combination with one or more selected from minoxidil, finasteride, dutasteride and another azole compound, to the scalp of a human subject who prior to administration exhibits hair loss and/or thinning on the scalp.

### Example 8

A formulation of econazole is topically administered at least once per day for up to six months to the scalp of a human subject who prior to administration exhibits hairline recession (such as, but not limited to, bitemporal recession) on the scalp.

### Example 9

A formulation of prochloraz is topically administered at least once per day for up to six months to the scalp of a human subject who prior to administration exhibits hairline recession (such as, but not limited to, bitemporal recession) on the scalp.

### Example 10

A formulation of econazole and prochloraz is topically administered at least once per day for up to six months to the scalp of a human subject who prior to administration exhibits hairline recession (such as, but not limited to, bitemporal recession) on the scalp.

### Example 11

A formulation of econazole is topically administered at least once per day for up to six months, in combination with one or more selected from minoxidil, finasteride, dutasteride and another azole compound, to the scalp of a human subject who prior to administration exhibits hairline recession (such as, but not limited to, bitemporal recession) on the scalp.

### Example 12

A formulation of prochloraz is topically administered at least once per day for up to six months, in combination with one or more selected from minoxidil, finasteride, dutasteride and another azole compound, to the scalp of a human subject who prior to administration exhibits hairline recession (such as, but not limited to, bitemporal recession) on the scalp.

### Example 13

A formulation of econazole and prochloraz is topically administered at least once per day for up to six months, in combination with one or more selected from minoxidil, finasteride, dutasteride and another azole compound, to the scalp of a human subject who prior to administration exhibits hairline recession (such as, but not limited to, bitemporal recession) on the scalp.

### Example 14

A formulation of econazole is topically administered at least once per day for up to six months to the scalp of a human subject who prior to administration exhibits lack of hair growth on the scalp.

### Example 15

A formulation of prochloraz is topically administered at least once per day for up to six months to the scalp of a human subject who prior to administration exhibits lack of hair growth on the scalp.

### Example 16

A formulation of econazole and prochloraz is topically administered at least once per day for up to six months to the scalp of a human subject who prior to administration exhibits lack of hair growth on the scalp.

### Example 17

A formulation of econazole is topically administered at least once per day for up to six months, in combination with one or more selected from minoxidil, finasteride, dutasteride and another azole compound, to the scalp of a human subject who prior to administration exhibits lack of hair growth on the scalp.

### Example 18

A formulation of prochloraz is topically administered at least once per day for up to six months, in combination with one or more selected from minoxidil, finasteride, dutasteride and another azole compound, to the scalp of a human subject who prior to administration exhibits lack of hair growth on the scalp.

### Example 19

A formulation of econazole and prochloraz is topically administered at least once per day for up to six months, in combination with one or more selected from minoxidil, finasteride, dutasteride and another azole compound, to the scalp of a human subject who prior to administration exhibits lack of hair growth on the scalp.

### Example 20

A formulation of econazole is topically administered at least once per day for up to six months to the skin of a human subject who prior to administration exhibits acne, seborrheic dermatitis, or one or more symptoms of skin aging, or any combination thereof.

### Example 21

A formulation of prochloraz is topically administered at least once per day for up to six months to the skin of a human subject who prior to administration exhibits acne, seborrheic dermatitis, or one or more symptoms of skin aging, or any combination thereof.

### Example 22

A formulation of econazole and prochloraz is topically administered at least once per day for up to six months to the skin of a human subject who prior to administration exhibits acne, seborrheic dermatitis, or one or more symptoms of skin aging, or any combination thereof.

### Example 23

A formulation of econazole is topically administered at least once per day for up to six months, in combination with one or more selected from minoxidil, finasteride, dutasteride and another azole compound, to the skin of a human subject who prior to administration exhibits acne, seborrheic dermatitis, or one or more symptoms of skin aging, or any combination thereof.

### Example 24

A formulation of prochloraz is topically administered at least once per day for up to six months, in combination with one or more selected from minoxidil, finasteride, dutasteride and another azole compound, to the skin of a human subject who prior to administration exhibits acne, seborrheic dermatitis, or one or more symptoms of skin aging, or any combination thereof.

### Example 25

A formulation of econazole and prochloraz is topically administered at least once per day for up to six months, in combination with one or more selected from minoxidil, finasteride, dutasteride and another azole compound, to the skin of a human subject who prior to administration exhibits acne, seborrheic dermatitis, or one or more symptoms of skin aging, or any combination thereof.

### EMBODIMENTS

Embodiment 1. A method of treating androgenic alopecia in a subject in need thereof, the method comprising administering to the subject a therapeutically effective amount of econazole, prochloraz, or a combination thereof.

Embodiment 2. A method of reducing or stopping hair thinning in a subject in need thereof, the method comprising administering to the subject a therapeutically effective amount of econazole, prochloraz, or a combination thereof.

Embodiment 3. A method of reversing hair thinning in a subject in need thereof, the method comprising administering to the subject a therapeutically effective amount of econazole, prochloraz, or a combination thereof.

Embodiment 4. A method of promoting hair growth in a subject in need thereof, the method comprising administering to the subject a therapeutically effective amount of econazole, prochloraz, or a combination thereof.

Embodiment 5. The method of any one of embodiments 1-4, wherein econazole, prochloraz, or the combination thereof is administered topically on a scalp of the subject.

Embodiment 6. A method of reversing hairline recession in a subject in need thereof, the method comprising administering to the subject a therapeutically effective amount of econazole or prochloraz, or a combination thereof.

Embodiment 7. The method of embodiment 6, wherein the method includes reversing bitemporal recession.

Embodiment 8. The method of embodiment 6 or embodiment 7, wherein econazole, prochloraz, or the combination thereof is administered topically at an area of scalp with hairline recession.

Embodiment 9. The method of embodiment 5 or embodiment 8, wherein the scalp is shaved prior to administration of econazole, prochloraz, or the combination thereof.

Embodiment 10. The method of embodiment 5 or embodiment 8, wherein the scalp is shaved 2-3 times weekly for 4 to 6 weeks upon commencement of administration of econazole, prochloraz, or the combination thereof.

Embodiment 11. The method of any one of embodiments 1-10, wherein the subject is a human.

Embodiment 12. The method of any one of embodiments 1-10, wherein the subject is a non-human mammal.

Embodiment 13. A method of treating a skin condition in a subject in need thereof, the method comprising administering to the subject a therapeutically effective amount of econazole or prochloraz, or a combination thereof.

Embodiment 14. The method of embodiment 13, wherein the skin condition is skin aging or one or more selected from the group consisting of dry skin, psoriasis, eczema, acne, seborrheic dermatitis, wrinkling, and loss of collagen.

Embodiment 15. The method of embodiment 13 or embodiment 14, wherein econazole, prochloraz, or the combination thereof is administered topically at an area of skin with skin aging or one or more selected from the group consisting of dry skin, psoriasis, eczema, acne, seborrheic dermatitis, wrinkling, and loss of collagen.

Embodiment 16. The method of embodiment 15, wherein the area of skin is located at the face, neck, chest, back, or any combination of two or more thereof.

Embodiment 17. The method of any one of embodiments 1-16, wherein econazole, prochloraz, or the combination thereof is administered as a formulation selected from a cream, ointment, lotion, serum, foam, gel, paste, aerosol, powder, and liquid.

Embodiment 18. The method of embodiment 17, wherein econazole, prochloraz, or the combination thereof, is present in the formulation in an amount of about 0.1 wt. % to about 10 wt. %.

Embodiment 19. The method of any one of embodiments 1-18, wherein econazole, prochloraz, or the combination thereof are formulated for administration through immersion of, bathing, or soaking the subject.

Embodiment 20. The method of any one of embodiments 1-19, wherein econazole, prochloraz, or the combination thereof is administered at least once per day.

Embodiment 21. The method of any one of embodiments 1-20, wherein econazole, prochloraz, or the combination thereof is administered once or twice per day

Embodiment 22. The method of any one of embodiments 1-21, wherein econazole, prochloraz, or the combination thereof is administered in combination with one or more additional therapeutic agents.

Embodiment 23. The method of embodiment 22, wherein the one or more additional therapeutic agents are selected from minoxidil, finasteride, dutasteride, fenugreek, another azole compound, and any combination of two or more thereof.

Embodiment 24. The method of embodiment 22 or embodiment 23, wherein econazole, prochloraz, or the combination thereof and the one or more additional therapeutic agents are administered concurrently.

Embodiment 25. The method of embodiment 22 or embodiment 23, wherein econazole, prochloraz, or the combination thereof and the one or more additional therapeutic agents are administered sequentially.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A method of treating androgenic alopecia in a subject in need thereof, the method comprising administering to the subject a therapeutically effective amount of econazole, prochloraz, or a combination thereof.

2. A method of reducing or stopping hair thinning in a subject in need thereof, the method comprising administering to the subject a therapeutically effective amount of econazole, prochloraz, or a combination thereof.

3. A method of reversing hair thinning in a subject in need thereof, the method comprising administering to the subject a therapeutically effective amount of econazole, prochloraz, or a combination thereof.

4. A method of promoting hair growth in a subject in need thereof, the method comprising administering to the subject a therapeutically effective amount of econazole, prochloraz, or a combination thereof.

5. The method of claim 1, wherein
econazole, prochloraz, or the combination thereof is administered topically on a scalp of the subject.

6. The method of claim 5, wherein the scalp is shaved prior to administration of econazole, prochloraz, or the combination thereof.

7. The method of claim 5, wherein the scalp is shaved 2-3 times weekly for 4 to 6 weeks upon commencement of administration of econazole, prochloraz, or the combination thereof.

8. The method of claim 1, wherein the subject is a human.

9. The method of claim 1, wherein the subject is a non-human mammal.

10. A method of reversing hairline recession in a subject in need thereof, the method comprising administering to the subject a therapeutically effective amount of econazole or prochloraz, or a combination thereof.

11. The method of claim 10, wherein the method includes reversing bitemporal recession.

12. The method of claim 10, wherein econazole, prochloraz, or the combination thereof is administered topically at an area of scalp with hairline recession.

13. The method of claim 12, wherein the scalp is shaved prior to administration of econazole, prochloraz, or the combination thereof.

14. The method of claim 12, wherein the scalp is shaved 2-3 times weekly for 4 to 6 weeks upon commencement of administration of econazole, prochloraz, or the combination thereof.

15. A method of treating a skin condition in a subject in need thereof, the method comprising administering to the subject a therapeutically effective amount of econazole or prochloraz, or a combination thereof.

16. The method of claim 15, wherein the skin condition is skin aging or one or more selected from the group consisting of dry skin, psoriasis, eczema, acne, seborrheic dermatitis, wrinkling, and loss of collagen.

17. The method of claim 15, wherein econazole, prochloraz, or the combination thereof is administered topically at an area of skin with skin aging or one or more selected from the group consisting of dry skin, psoriasis, eczema, acne, seborrheic dermatitis, wrinkling, and loss of collagen.

18. The method of claim 17, wherein the area of skin is located at the face, neck, chest, back, or any combination of two or more thereof.

19. The method of claim 1, wherein econazole, prochloraz, or the combination thereof is administered as a formulation selected from a cream, ointment, lotion, serum, foam, gel, paste, aerosol, powder, and liquid.

20. The method of claim 19, wherein econazole, prochloraz, or the combination thereof, is present in the formulation in an amount of about 0.1 wt. % to about 10 wt. %.

21. The method of claim 1, wherein econazole, prochloraz, or the combination thereof are formulated for administration through immersion of, bathing, or soaking the subject.

22. The method of claim 1, wherein econazole, prochloraz, or the combination thereof is administered at least once per day.

23. The method of claim 1, wherein econazole, prochloraz, or the combination thereof is administered once or twice per day

24. The method of claim 1, wherein econazole, prochloraz, or the combination thereof is administered in combination with one or more additional therapeutic agents.

25. The method of claim 24, wherein the one or more additional therapeutic agents are selected from minoxidil, finasteride, dutasteride, fenugreek, another azole compound, and any combination of two or more thereof.

26. The method of claim 24, wherein econazole, prochloraz, or the combination thereof and the one or more additional therapeutic agents are administered concurrently.

27. The method of claim 24, wherein econazole, prochloraz, or the combination thereof and the one or more additional therapeutic agents are administered sequentially.
